(19) United States
(12) Patent Application Publication
(10) Pub. No.: US 2026/0325777 A1
(43) Pub. Date: Oct. 22, 2026

(54) Oral Airway Assemblies and Methods

(71) Applicant: NUZONE MEDICAL LLC, Portland, OR (US)

(72) Inventors: Beata Zawadzka, Portland, OR (US); Daniel Dean Ogilvie, Spokane, WA (US)

(73) Assignee: NUZONE MEDICAL LLC, Portland, OR (US)

(21) Appl. No.: 19/655,114

(22) Filed: Apr. 22, 2026

Related U.S. Application Data
(60) Provisional application No. 63/792,525, filed on Apr. 22, 2025.

(51) Int. Cl. A61M 16/04; A61M 16/00; A61M 16/20

(52) U.S. Cl. CPC A61M 16/049; A61M 16/0003; A61M 16/024; A61M 16/208; A61M 2202/0007; A61M 2202/02; A61M 2205/0227; A61M 2205/3313; A61M 2205/502; A61M 2205/584; A61M 2205/8206; A61M 2210/0625; A61M 2230/43

(57) ABSTRACT

An oral airway assembly is provided that includes a flange that incorporates processing circuitry to determine component levels of air passing through the oral assembly. A method of monitoring air components during airway management is provided. The method comprises: inserting an oral airway assembly into a patient's mouth, the oral airway assembly comprising a body with an opening for air passage and an air component indicator associated with the opening; allowing air to pass through the opening during patient respiration or ventilation; and observing an indication from the air component indicator to determine the presence or level of at least one air component in the exhaled or passing air.

### CROSS REFERENCE TO RELATED APPLICATION

This application claims priority to and the benefit of U.S. Provisional Patent Application Ser. No. 63/792,525 Filed Apr. 22, 2025, entitled “Oral Airway Assemblies and Methods”, the entirety of which is incorporated by reference herein.

### TECHNICAL FIELD

The technical field is oral airway assemblies and methods, particularly oral airway assemblies and methods that include air component indicators.

### BACKGROUND

Breathing difficulties may occur from severe trauma (such as trauma that may result from a vehicular crash), medical conditions, drug reactions, smoke inhalation, etc. Professional medical emergency response personnel (for instance, paramedics, medevac crews, firefighters, etc.) are thus trained to provide breathing assistance to injured persons. The breathing assistance may include ventilation and oxygenation (with oxygenation comprising administration of oxygen-enriched gas mixtures).

The administration of the ventilation and oxygenation may involve placement of a mask over the injured person's mouth, and/or insertion of a cannula into the injured person's mouth. An oral airway device is often placed in an injured person's mouth prior to utilization of the mask and/or cannula to prevent the soft tissues of the oropharynx (i.e., the part of the throat at the back of the mouth) from collapsing into and obstructing the airway. This can be particularly important if the injured person is unconscious, or in danger of becoming unconscious.

Commercially available oral airway devices often comprise a region that rests between the top and bottom teeth, together with a region having a distal curve (i.e., the blade) to provide support along the back of the palate. The region resting between the teeth may be configured to prevent clenching of the teeth on the tongue, as well as to prevent clenching of the teeth on medical structures (such as, for example, an endotracheal tube, a suction catheter, a fiberoptic laryngoscope, etc.) which may be passed into the oropharynx while the oral airway device is in place.

A problem for medical emergency response personal (e.g., so-called “first responders”) is to find an appropriately sized oral airway device for the injured person (or to find appropriately sized devices for more than one injured person if multiple injured persons are present at a site). If the wrong sized oral airway device is inserted into a person, it may fail to restrain soft tissues (if it is too small for a person's mouth and oropharynx), or may gag and/or choke the person (if it is too large for the person's mouth and oropharynx). Presently, medical emergency response personnel may carry a wide selection of oral airway device sizes and shapes in order to be adequately prepared for numerous different sizes of injured persons. However, stocking of a selection of different sizes of oral airway devices consumes valuable space in a tool kit. It is desired to develop improved oral airway devices that could be used for persons of different sizes, so that a single device could substitute for numerous different sizes of devices.

There has been some effort to develop adjustable oral airway devices. In theory, adjustable oral airway devices could eliminate the need for medical emergency response personnel to stock numerous different sizes of oral airway devices. However, the adjustable oral airway devices utilized by medical emergency response personnel should be suitable for rapid deployment under the stress of emergency situations, and under the awkwardness of the difficult environment conditions that may be encountered by the personnel in emergency situations. In practice, the presently available adjustable oral airway devices tend to be unsuitable for utilization by medical emergency response personnel under the adverse, and time-sensitive, conditions that may be encountered by such personnel. Accordingly, it is desired to develop improved adjustable oral airway devices.

### SUMMARY

An oral airway assembly is provided that includes a flange that incorporates processing circuitry to determine component levels of air passing through the oral assembly.

A method of monitoring air components during airway management is provided. The method comprises: inserting an oral airway assembly into a patient's mouth, the oral airway assembly comprising a body with an opening for air passage and an air component indicator associated with the opening; allowing air to pass through the opening during patient respiration or ventilation; and observing an indication from the air component indicator to determine the presence or level of at least one air component in the exhaled or passing air.

### BRIEF DESCRIPTION OF DRAWINGS

Embodiments of the disclosure are described below with reference to the following accompanying drawings.

FIG. 1 is a depiction of an oral airway assembly according to an embodiment of the disclosure.

FIG. 2A is a depiction of an oral airway assembly according to another embodiment of the disclosure.

FIG. 2B is a depiction of an oral airway assembly according to another embodiment of the disclosure.

FIG. 3 is a depiction of a front view of the oral airway assembly of FIG. 2.

FIG. 4 is a depiction of at least one cross sectional view of the oral airway assembly of FIG. 2.

FIG. 5 is a depiction of an oral airway assembly according to another embodiment of the disclosure.

FIG. 6 is a depiction of the oral airway assembly of FIG. 5 indicating the presence of an air compound.

FIG. 7 is a depiction of a portion of an oral airway assembly according to another embodiment of the disclosure.

FIG. 8 is another view of the airway assembly of FIG. 7.

FIG. 9 is a cross sectional view of the portion of the airway assembly of FIG. 7.

FIG. 10 is another view of the airway assembly of FIG. 7.

FIG. 11 is a depiction of the processing circuitry of the portion of the airway assembly of FIG. 7.

FIG. 12 is a schematic view of the processing circuitry of the airway assembly of FIG. 7.

### DESCRIPTION

This disclosure is submitted in furtherance of the constitutional purposes of the U.S. Patent Laws “to promote the progress of science and useful arts” (Article 1, Section 8).

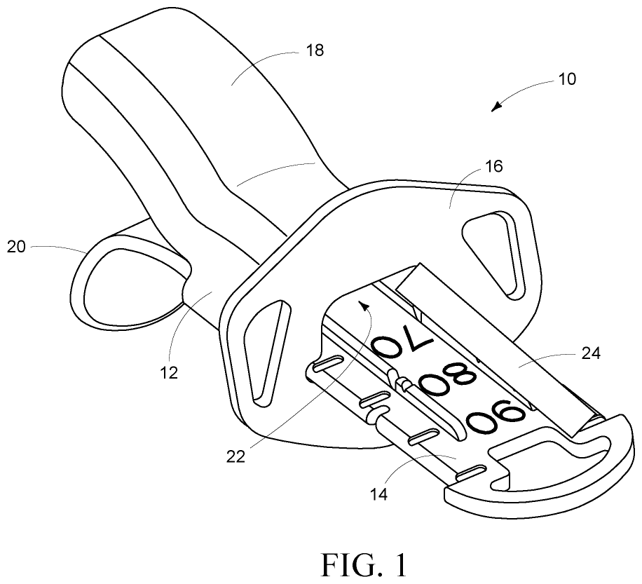

The present disclosure will be described with reference to FIGS. 1-12. Referring first to FIG. 1, and with reference to U.S. Pat. Nos. 8,297,275 and 8,931,477, the entirety of each of which are incorporated herein by reference, FIG. 1 depicts an oral airway assembly 10 that includes a body 12 and a lower member 14. As shown, lower member 14 can include an end 20 and there can be an opening 22 within body 12, and body 12 can extend to an upper member 18. This disclosure includes an air component indicator strip 24. Air component indicator strip 24 can be specifically designed to indicate components of air passing through opening 22 when inserted into a patient. Air component indicator strip 24 can be specific to CO₂, for example, and indicate certain levels of CO₂ as air passes over the indicator strip. Other indicator strips can be utilized that indicate other components of air as well.

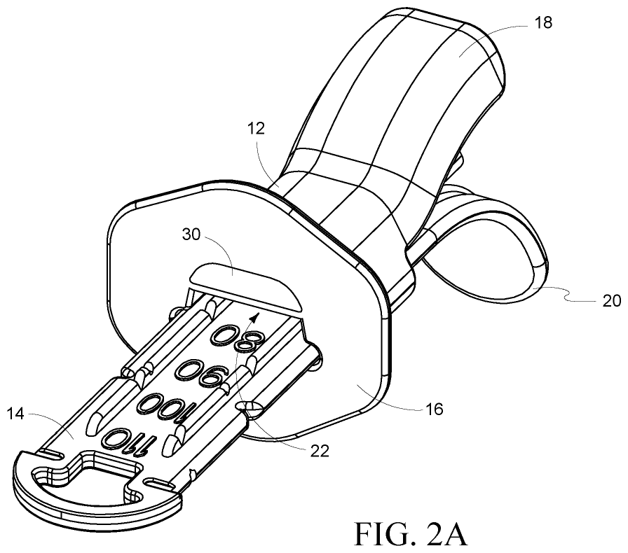

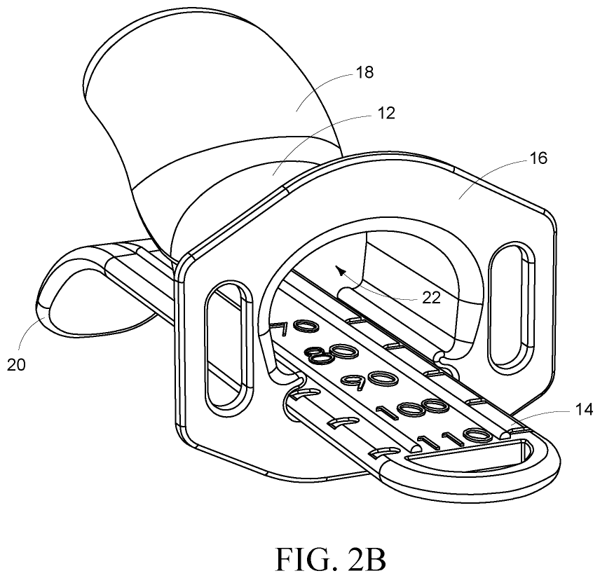

Referring next to FIGS. 2A and 2B, within body 12 can be an additional opening 30 that extends through body 12 and is available to receive and convey air from and to the patient while allowing for the lower member to be used as a guide for a breathing tube, if necessary. As shown in FIGS. 2A and 2B opening 22 can be of different sizes. In the embodiment of FIG. 2B, opening 22 can be large enough to accommodate medical devices used during scoping procedures (i. e bronchoscopy, endoscopy, etc.). Accordingly, the opening of FIG. 2B is of a different shape and size, and is to configured to maintain an patent airway in addition to allow the scope to safely pass through the oropharyngeal cavity.

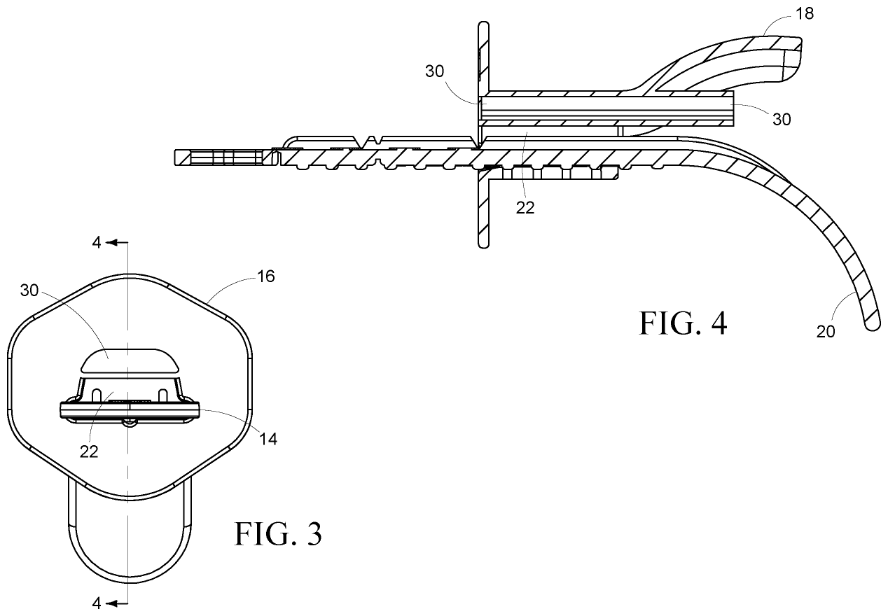

Referring next to FIG. 3, in another detail, opening 22, flange 16, as well as lower member 14 are shown in relation to opening 30. In a cross section detailed in FIG. 4, opening 30 is shown to extend beyond flange 16 and to the patient below the upper member 18.

Referring next to FIG. 5, an indicator strip 50 is placed proximate or in operational arrangement with opening 30 to receive air being conveyed to and from the patient. In FIG. 5, the indicator strip can indicate a component of air at a certain level, and in FIG. 6, indicator strip 52 can indicate a component of air at another level. In at least one example, the indicator strip can be purple at one level and yellow at another. In accordance with example implementations, these can be color coded indicator strips or indicator housings within this arrangement.

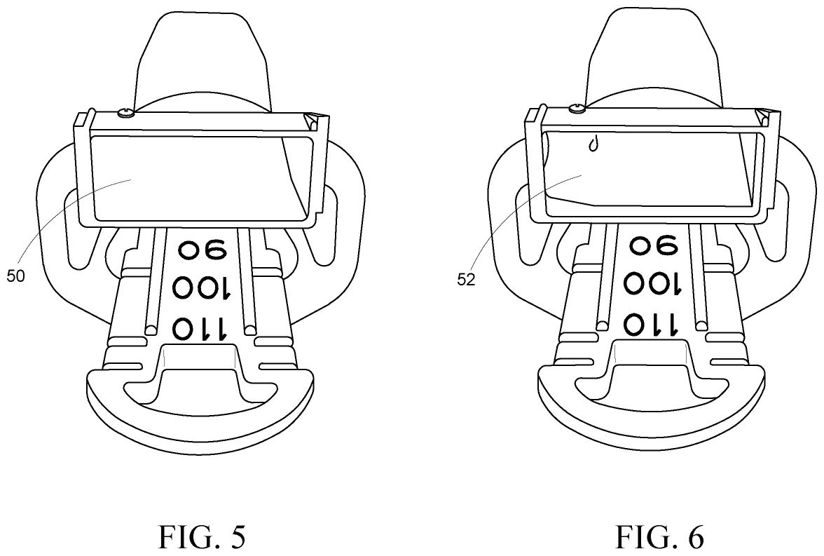

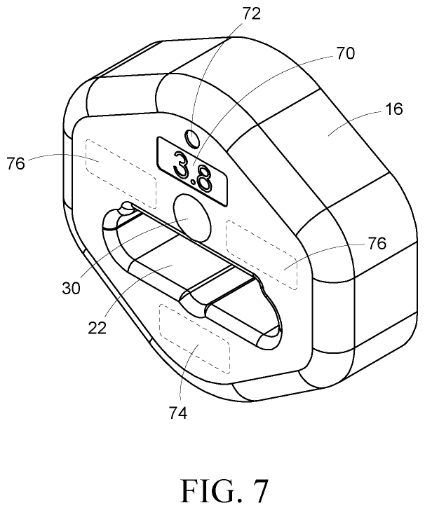

Referring next to FIG. 7, flange 16 of the body is shown developed to include operational circuitry or processing circuitry in combination with opening 30 that can or cannot be sealed by a poppet valve which can receive or open upon exhale. Flange 16 can include a visual indicator 72 as well as a digital interface 70 in combination with sensor 76 and power source 74. Visual indicator 72 can be configured to provide a specific colored light that indicates the presence of an air compound of a specific amount; for example, yellow at one level and green at another. Sensor 76 can be arranged in combination with a one-way valve associated with opening 30. This one-way valve is shown in FIG. 9 as 90. Sensor 76 can be in communication with power source 74 as well as interface 70. Sensor 76 can be, for example, UV sensors that can pass through opening 30 and determine the CO₂ content of the air that is in opening 30. The power source 74 can be rechargeable, plug-in, and/or a small lithium battery source, for example.

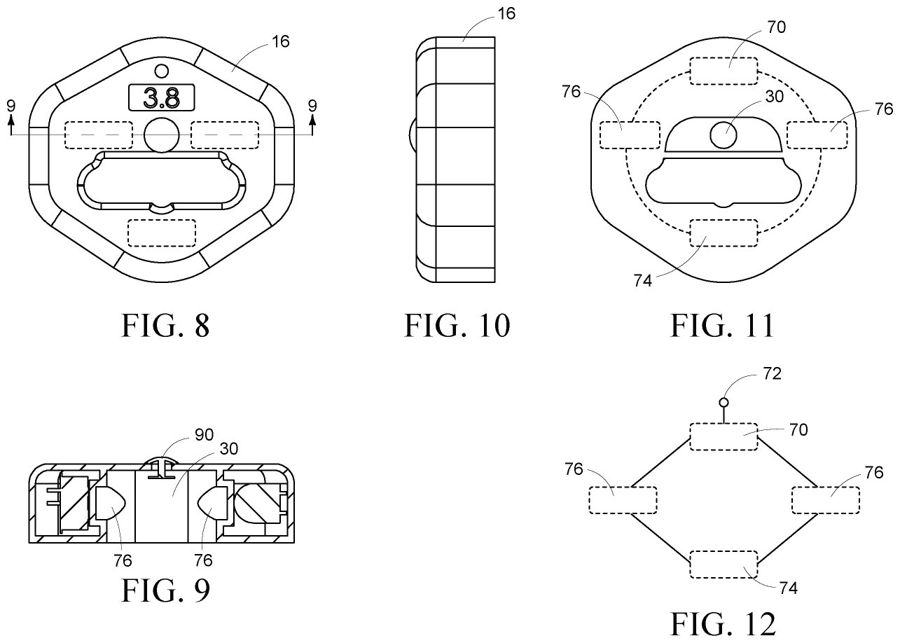

As shown in FIG. 8, a front depiction of flange 16 is shown with a cross-section 9. Cross-section 9 indicates sensor 76 for the determination of CO₂ in opening 30. As can be seen from the drawings in FIGS. 10-12, the processing circuitry 70, 72, 76 and 74 is shown as operably aligned.

In compliance with the statute, embodiments of the invention have been described in language more or less specific as to structural and methodical features. It is to be understood, however, that the entire invention is not limited to the specific features and/or embodiments shown and/or described, since the disclosed embodiments comprise forms of putting the invention into effect.

The invention claimed is:

1. An oral airway assembly that includes a flange that incorporates processing circuitry to determine component levels of air passing through the oral assembly.

2. The oral airway assembly of claim 1 further comprising:
a body having a proximal end and a distal end, the body including the flange at the proximal end and an upper member extending distally from the flange;
a lower member coupled to the body and configured to be adjustably positioned relative to the upper member;
an opening extending through the body and configured to allow passage of air to and from a patient when the oral airway assembly is inserted in the patient's mouth; and
an air component indicator associated with the opening and configured to indicate the presence or level of at least one air component in air passing through the opening.

3. The oral airway assembly of claim 2, wherein the air component indicator comprises a chemical indicator strip positioned to contact air flowing through the opening, the chemical indicator strip configured to undergo a visible color change in response to a predetermined level of the at least one air component.

4. The oral airway assembly of claim 3, wherein the at least one air component is carbon dioxide (CO₂), and the chemical indicator strip is configured to change from a first color to a second color when the CO₂ level exceeds a threshold.

5. The oral airway assembly of claim 2, further comprising a second opening extending through the body, wherein the air component indicator is positioned proximate or within the second opening.

6. The oral airway assembly of claim 2, wherein the air component indicator comprises:
a sensor arranged to detect the at least one air component in air within or passing through the opening;
processing circuitry in communication with the sensor; and
a visual indicator operably coupled to the processing circuitry and configured to provide a visible signal corresponding to a detected level of the at least one air component.

7. The oral airway assembly of claim 6, wherein the visual indicator is configured to emit light of a first color at a first detected level of the air component and light of a second color at a second detected level of the air component.

8. The oral airway assembly of claim 6, wherein the sensor comprises a UV sensor configured to determine CO₂ content.

9. The oral airway assembly of claim 6, further comprising a power source coupled to the processing circuitry and sensor, the power source selected from the group consisting of a rechargeable battery, a plug-in power source, and a lithium battery.

10. The oral airway assembly of claim 6, further comprising a one-way valve associated with the opening, wherein the sensor is arranged in operational communication with the one-way valve.

11. The oral airway assembly of claim 6, further comprising a digital interface operably coupled to the processing circuitry and configured to display information related to the detected level of the air component.

12. The oral airway assembly of claim 2, wherein the lower member includes a distal end configured to provide support along the back of the palate, and the body is configured such that the lower member can be used as a guide for a breathing tube.

13. A method of monitoring air components during airway management, the method comprising:
inserting an oral airway assembly into a patient's mouth, the oral airway assembly comprising a body with an opening for air passage and an air component indicator associated with the opening;
allowing air to pass through the opening during patient respiration or ventilation; and
observing an indication from the air component indicator to determine the presence or level of at least one air component in the exhaled or passing air.

14. The method of claim 13, wherein the air component indicator comprises a color-changing chemical strip, and observing comprises visually detecting a color change of the strip.

15. The method of claim 13, wherein the air component indicator comprises a sensor and visual indicator, and observing comprises viewing a colored light or digital display generated by the visual indicator in response to sensor output.

16. The oral airway assembly of claim 2, wherein the at least one air component is carbon dioxide.
